(19) United States
(12) Patent Application Publication
(10) Pub. No.: US 2013/0023560 A1
(43) Pub. Date: Jan. 24, 2013

(54) METHODS AND COMPOSITIONS FOR THE TREATMENT OF IRON TOXICITY

(71) Applicants: Christopher S. Wilcox, Washington, DC (US); Pouneh Nouri, Washington, DC (US)

(72) Inventors: Christopher S. Wilcox, Washington, DC (US); Pouneh Nouri, Washington, DC (US)

(73) Assignee: Georgetown University, Washington, DC (US)

(21) Appl. No.: 13/425,036

(22) Filed: Mar. 20, 2012

Related U.S. Application Data
(63) Continuation of application No. 12/566,377, filed on Sep. 24, 2009, now abandoned.
(60) Provisional application No. 61/100,421, filed on Sep. 26, 2008.

(51) Int. Cl. A61K 31/445; A61P 3/00; A61K 31/195

(52) U.S. Cl. 514/315; 514/562

(57) ABSTRACT

Methods and compositions for the treatment of iron toxicity using nitroxides, particularly 4-hydroxy-2,2,6,6-tetramethyl-1-piperidine-1-oxyl (Tempol), N-acetylcysteine (NAC), and combinations thereof.

### CROSS-REFERENCE TO RELATED APPLICATIONS

This application claims priority to U.S. Provisional Patent Application No. 61/100,421, filed on Sep. 28, 2009, currently pending and hereby incorporated by reference in their entirety

### BACKGROUND OF THE INVENTION

The invention relates generally to methods and compositions for the treatment of iron toxicity by administering nitroxides, such as 4-hydroxy-2,2,6,6-tetramethyl-1-piperidine-1-oxyl (Tempol), N-acetylcysteine (NAC), and/or combinations thereof.

Iron is essential to most life forms and to normal human physiology. In humans, iron is an essential component of oxygen transport and the regulation of cell growth and differentiation.

Under normal conditions, most iron in the body is tightly bound either to functional molecules such as hemoglobin, to transport proteins such as transferrin, or to intracellular storage proteins such as ferritin (Hershko et al., Non-transferrin plasma iron. Br J Haematol. 1987;66(2)):149-51.

Regulation of iron in this manner is important, as unbound, or free, iron is highly damaging to cells and tissues. For example, parenteral iron preparations have been shown to increase biological markers of oxidative stress in cell cultures, animal models, and in hemodialysis patients, including those suffering from end stage renal disease (ESRD). (al., 1999) (al, 2007 Jun. 22), (al Z. P., 2007 Mar;27(3)), (Agarwal, 2006), (al R. a., 2004), (Besarab, 1999), (Zager, 2006).

It is believed that the labile iron pool (LIP), or non-transferrin-bound iron, is responsible for the demonstrated increase in oxidative stress (al e. B., 2002 Suppl) by exerting its pro-oxidant effect through the Fenton reaction (Cavill, 2003 Suppl 8):

Fe²⁺+H₂O₂→Fe³⁺+OH·+OH⁻

As red blood cells degrade, they release free iron, which is then gradually redistributed through normal homeostatic mechanisms. However, disruption or overload of the body's iron regulatory mechanisms can result in excess accumulation of iron, which is stored as hemosiderin. Over time, this excess accumulation becomes observable as a slate-gray hyperpigmentation of the skin.

There are several known sources of excess iron accumulation. For example, hemochromatosis is an inherited disorder characterized by excessive absorption of dietary iron and hemosiderosis, or transfusional iron overload, stems from the degradation of transfused red blood cells that in turn results in iron levels that exceed normal homeostatic mechanisms.

Intravenous iron supplementation is another source of excess iron accumulation, and is often used to treat anemics and/or those suffering from ESRD (in fact, anemia commonly accompanies ESRD). With respect to anemia, a functional iron deficiency arises from either erythropoietin deficiency and/or insufficient dietary iron absorption in most cases. This deficiency may also occur or be exacerbated in those anemics treated with erythropoiesis stimulating agents (ESA). With respect to those suffering from ESRD, compromised kidney function often necessitates hemodialysis, which in turn often further necessitates intravenous iron supplementation. In fact, dialysis patients receive an average of 2.5 g of intravenous iron annually and dialysis patients are at higher risk for cardiovascular diseases (CVD) including diabetes, hypertension, coronary artery disease and stroke which may be exacerbated by interaction with anemia, hypercalcemia, hypovitaminosis D, hyperhomocystemia, and hyperphosphatemia. Iron has been recognized as a risk factor for CVD in this population. (Kletzmay, 2002 Suppl 2).

Venous stasis disease, dermatitis, and ulceration are yet other diseases in which excess iron accumulation is an issue. Venous disease is quite common, and the conditions, ranging from simple varicose veins to deep venous thrombosis, that make up this broad category of disorders affect up to 2% of the population. While certain of these conditions merely reduce the quality of a patient's life, others can lead to death. Under certain circumstances, venous thrombosis is one of these latter conditions. Venous thromboses commonly fall into two types: superficial venous thrombosis and deep venous thrombosis.

Venous thrombosis may occur for several reasons, such as periods of prolonged inactivity. This reason is often identified as the culprit in otherwise normal subjects that have a prolonged airplane flight, in those whose leg is immobilized because of trauma, in those with heart failure, and in those confined to bed for extended lengths of time.

Venous thrombosis may also arise from local injury. Venous thrombosis is anticipated in a high proportion of patients admitted to hospital for surgical procedures on the lower limb or pelvis (for example, hip replacement).

A portion of patients (about one-third) diagnosed with venous thrombosis have an identified genetic predisposition to this condition. In these patients, one or more single nucleotide polymorphisms in genes result in defective proteins involved in blood coagulation (for example, Leiden factor, prothrombin, or prothrombin-activating or -inhibiting components). These defective proteins result in a hypercoagulable state of blood, which in turn increases the risk of a clot. Since these predisposing factors are sometimes irreversible, and since prior thrombosis and damage to veins predispose a person to repeat thromboses, many patients suffer multiple episodes of venous thrombosis.

A venous thrombosis in the lower limb typically presents with a relatively acute development of edema, pain, and erythema affecting the calf and ankle Venous thrombosis complicates muscle strains, ligament damage or other trauma to the lower limb and can also complicate other hematological disorders such as polycythemia. Untreated, a venous thrombosis may result in a pulmonary embolism, which can be fatal.

In the majority of patients, especially those treated with anti-coagulants, a venous thrombosis resolves itself over time and local symptoms disappear over a course of days or weeks as recannulization of veins takes place.

Unfortunately, in a percentage of patients who have had a single clot, and in a high proportion of those who have recurrent clots, there is sufficient damage to the valves of the veins during the recannulization process as to impair venous function.

In the case of a deep vein thrombosis, the valves in the affected veins may become injured, scarred, and/or thickened, preventing them from closing properly and maintaining normal cephalad blood flow. Secondary changes in the vein, such as hypertrophy, tortuosity and further dilation may result. This venous incompetence leads to an increase in hydrostatic pressure, which is worsened when the legs are in a dependent position (e.g., standing upright). Blood pools in the veins, resulting in edema and dull ache around the ankle and lower leg.

A prolonged increase in pressure within in the small veins at the ankle may lead to a loss of proper function of the veins in the legs that normally carry blood back to the heart, a condition known as venous stasis. Venous stasis typically first manifests as persistent edema and irritation of the skin around the medial or lateral malleoli. If the swelling is not controlled, inflammation occurs in the skin and the tissues beneath the skin in the area of the ankle

A prolonged increase in pressure within the veins has further adverse effects, the mechanism of which are not fully understood. Any increase in pressure in the venules in the skin is associated with a local axonal reflex which increases vasoconstriction of the arterioles to that region. This is a protective reflex to prevent excessive pressure at the capillaries thereby preventing capillary damage and edema. However, this reflex vasoconstriction reduces tissue blood flow and predisposes the skin to ischemia and ulceration.

Distension of blood vessels, as occurs in veins during venous stasis also leads to superoxide formation in blood vessels and surrounding tissues, and increases their permeability. Both superoxide and hydroxyl radicals exacerbate the vasoconstriction and may thereby further contribute to skin ischemia and ulceration. The increase in blood vessel permeability also exacerbates edema and may lead to extravasation of plasma and blood constituents, which in turn results in excess iron accumulation from the breakdown of red blood cells.

Irrespective of the particular source, excess iron accumulation leads to increased oxidative stress and eventual cellular and tissue damage. In blood vessels, this excess oxidative stress leads to impaired endothelial-derived nitric oxide (NO) bioactivity that may cause impaired endothelial-dependent vaso-relaxation and lead to local vasoconstriction and ulceration. This process is exacerbated by the formation of peroxynitrite from the reaction between NO and the hydroxyl radical or superoxide.

Peroxynitrite can nitrosate proteins and alter their function. For example, nitrosation of prostacyclin synthase in the endothelium inactivates the enzyme and thereby reduces the generation of prostacyclin. Prostacyclin is a component of the endothelium-dependent relaxation response. Nitric oxide and prostacyclin are both released from the normal, healthy vascular endothelium and act together to maintain tissue blood flow, tissue integrity and to prevent platelet adhesion and thrombosis (Ullrich, V. and Bachschmidt, M., Superoxide as a Messenger of Endothelial Function. Biochem, Biophys, Res., Commune, 278: 1-8-2000).

Intravenous iron, as iron released into the skin, may simultaneously prevent the formation of bioreactive NO and prostacyclin. Excess formation of superoxide and hydroxyl radicals that reduce NO and prostacyclin are predicted to lead to local vasoconstriction, ischemia, tissue damage and microvascular thrombosis that precedes and worsen-established ulceration. Moreover, a similar process occurring in the circulation after intravenous iron therapy can lead to endothelial dysfunction. This is important since endothelial dysfunction is a predictor of cardiovascular morbidity and mortality in patients with cardiovascular disease or end-stage renal disease.

Excess hydroxyl radicals produced through the Fenton reaction also initiate fibrotic and sclerotic processes that impair normal skin function and healing. In venous stasis, these processes can produce a venous ulcer or chronic sore that develops on the inside of the ankle or on the surface of the ankle from a break in the skin. While venous stasis ulceration can be linked to a deposition of iron in the skin, a similar process may accompany other forms of dermal and mucosal ulceration. A general feature of ulceration is necrosis, leading to damage to blood vessels with escape of red blood cells into the tissues. This leads to bruising and discoloration of the site. Breakdown of red blood cells releases hemoglobin with further metabolism to hemosiderin and deposition of free iron. Therefore, local iron accumulation at the site of ulceration in the skin is likely a general phenomenon that predisposes to progressive ischemia, worsening of the ulceration and failure of therapeutic interventions.

Many ulcers are also associated with disease states other than venous disease. For example, cutaneous ulcers are associated with peripheral arterial and embolic disease, leukocytoclastic vasculitis, hemoglobinopathies such as sickle cell disease, cryoglobulinemia, cholesterol emboli, necrobiosis lipoidica, antiphospholipid syndrome, neuropathies, panniculitis, Raynaud's phenomenon, pyoderma gangrenosum, calciphylaxis, infection such as dimorphic fungi, chronic herpes varicella-Zoster lymphoma, Behcet's syndrome, erythema multiforme, primary blistering disorders, lupus erythematosus and bowel disease. Both cutaneous and mucocutaneous ulcers are associated with later stages of venous disease.

There are also multiple causes of ulcers. For example, the ulcer may be initiated by trauma, in which case there is quite clearly escape of blood into the skin. Alternatively, many ulcers are initiated by local ischemia, for example, in Raynaud's phenomenon or scleroderma, or sickle cell disease. Here, intense ischemia may be sufficient to damage vascular integrity and allow the escape of red blood cells. Other ulcers are associated with an increase of permeability which can be sufficient to allow the escape of blood constituents themselves into the interstitial space where a breakdown would release iron. Therefore, iron toxicity may be considered a general process contributing to skin ischemia, microvascular thrombosis, microvascular endothelial dysfunction, further escape of red blood cells, and the production of, and failure to heal, a skin or mucosal ulcer.

Once ulcers develop, they might take months to heal. In the case of venous ulcers, treatment includes meticulous wound care, elevation of the legs, compression stockings, and if necessary, antibiotics and other medications to rid the body of extra fluid. Sometimes even skin grafts are required, though most ulcers eventually heal without the need for surgical intervention. However, after healing, the ulcers will recur if swelling of the leg is not prevented.

Countering the pro-oxidant effect of parenteral iron has long been a subject of interest, and there is conflicting evidence that antioxidants may provide a solution. (al, 2007 Jun. 22), (al Z. P., 2007 March;27(3)), (Agarwal, 2006), (al R. a., 2004), (Besarab, 1999), (Zager, 2006).

For example, Vitamin C has not been shown to be effective in reducing iron-induced oxidative stress markers in the dialysis population, but rather to have a pro-oxidant effect when co-administered with iron in subjects with physiologic Vitamin C levels (al. E. J., 2006).

On the other hand, Vitamin E and selenium co-supplementation has been shown to be effective in reducing malondialdehyde (MDA) levels in dialysis patients when administered before intravenous iron (al A. M., 2007), but it is unclear if this co-supplementation affects other markers of oxidative stress.

In view of the lack of any conclusive evidence, there are currently no specific therapies or suggested solutions to address iron toxicity or prevent or ameliorate the damaging effects of hydroxyl radicals formed by free iron.

### BRIEF SUMMARY OF THE INVENTION

Disclosed herein are methods and compositions for the prevention and treatment of iron toxicity by administering a therapeutically effective amount of a nitroxide-containing composition, N-acetylcysteine (NAC)-containing composition, and/or combinations thereof. In some embodiments, the nitroxide is 4-hydroxy-2,2,6,6-tetramethyl-1-piperidine-1-oxyl (Tempol).

In certain embodiments, the composition is administered topically. In other embodiments, the compound is administered orally. In yet other embodiments, the compound is administered both topically and orally.

In certain embodiments, the methods and compositions are used to treat hemochromatosis. In other embodiments, the methods and compositions are used to treat or prevent hemosiderosis.

In certain embodiments, the methods and compositions are used to prevent the toxicity of iron required to treat anemia.

In certain embodiments, the methods and compositions may be co-administered with iron supplementation (e.g., intravenous iron supplementation). In other embodiments, the methods and compositions may be administered prior to, or subsequent to, iron supplementation (e.g., intravenous iron supplementation).

In certain embodiments, the methods and compositions are used to treat and prevent ulcers. In certain embodiments the ulcer is a cutaneous ulcer. In other embodiments, the ulcer is a mucocutaneous ulcer.

In particular embodiments, the cutaneous ulcer is an ulcer associated with one or more of the following conditions: peripheral arterial and embolic disease, leukocytoclastic vasculitis, hemoglobinopathies such as sickle cell disease, cryoglobulinemia, cholesterol emboli, necrobiosis lipoidica, antiphospholipid syndrome, neuropathies, panniculitis, Raynaud's phenomenon, pyoderma gangrenosum, calciphylaxis, infection from dimorphic fungi, chronic herpes varicella-Zoster lymphoma, Behcet's syndrome, erythema multiforme, primary blistering disorders, lupus erythematosus and inflammatory bowel disease, trauma, lymphatic obstruction, lymphangitis, infections of the skin, and venous stasis. In other particular embodiments, the dermal or mucocutaneous ulcer is an ulcer associated with trauma or venous stasis.

In certain embodiments, the cutaneous ulcer is treated by the application of a therapeutically effective amount of a topically administered hydroxy-2,2,6,6-tetramethyl-1-piperidine-1-oxyl-containing composition. This may also be applied to patients at risk of developing ulceration, for example those patients with venous stasis dermatitis who do not yet have a cutaneous ulcer.

In certain embodiments, the cutaneous ulcer is prevented or treated by the application of a therapeutically effective amount of a topically administered N-acetylcysteine-containing composition.

In certain embodiments, the cutaneous ulcer is prevented or treated by the application of a therapeutically effective amount of a topically administered combination of a hydroxy-2,2,6,6-tetramethyl-1-piperidine-1-oxyl-containing composition and a N-acetylcysteine-containing composition.

In certain embodiments, the mucocutaneous ulcer is prevented or treated by the application of a therapeutically effective amount of a topically administered hydroxy-2,2,6,6-tetramethyl-1-piperidine-1-oxyl-containing composition.

In certain embodiments, the mucocutaneous ulcer is prevented or treated by the application of a therapeutically effective amount of a topically administered N-acetylcysteine-containing composition.

In certain embodiments, the mucocutaneous ulcer is prevented or treated by the application of a therapeutically effective amount of a topically administered combination of a hydroxy-2,2,6,6-tetramethyl-1-piperidine-1-oxyl-containing composition and a N-acetylcysteine-containing composition.

In certain embodiments, the cutaneous ulcer is prevented or treated by the application of a therapeutically effective amount of an orally administered hydroxy-2,2,6,6-tetramethyl-1-piperidine-1-oxyl-containing composition.

In certain embodiments, the cutaneous ulcer is prevented or treated by the application of a therapeutically effective amount of an orally administered N-acetylcysteine-containing composition.

In certain embodiments, the cutaneous ulcer is prevented or treated by the application of a therapeutically effective amount of an orally administered combination of a hydroxy-2,2,6,6-tetramethyl-1-piperidine-1-oxyl-containing composition and a N-acetylcysteine-containing composition.

In certain embodiments, the mucocutaneous ulcer is prevented or treated by the application of a therapeutically effective amount of an orally administered hydroxy-2,2,6,6-tetramethyl-1-piperidine-1-oxyl-containing composition.

In certain embodiments, the mucocutaneous ulcer is prevented or treated by the application of a therapeutically effective amount of an orally administered N-acetylcysteine-containing composition.

In certain embodiments, the mucocutaneous ulcer is prevented or treated by the application of a therapeutically effective amount of an orally administered combination of a hydroxy-2,2,6,6-tetramethyl-1-piperidine-1-oxyl-containing composition and a N-acetylcysteine-containing composition.

In certain embodiments, the therapeutically effective amount of hydroxy-2,2,6,6-tetramethyl-1-piperidine-1-oxyl-containing composition ranges from 1 to 100 mg/kg/day.

### BRIEF SUMMARY OF THE SEVERAL VIEWS OF THE DRAWINGS

FIG. 1 illustrates the vasorelaxation response of the aortic ring to different doses of acetylcholine in rats previously administered saline or iron sucrose.

FIG. 2 illustrates the vasorelaxation response of the aortic ring to different doses acetylcholine in rats previously administered saline, iron sucrose, hydroxy-2,2,6,6-tetramethyl-1-piperidine-1-oxyl, or a combination of iron sucrose and hydroxy-2,2,6,6-tetramethyl-1-piperidine-1-oxyl (Tempol).

FIG. 3 illustrates the vasorelaxation response of the aortic ring to different doses acetylcholine in rats previously administered saline, iron sucrose, Vitamin C, or a combination of iron sucrose and Vitamin C.

FIG. 4 illustrates the vasorelaxation response of the aortic ring to different doses acetylcholine in rats previously administered saline, iron sucrose, or a combination of iron sucrose and N-acetylcysteine.

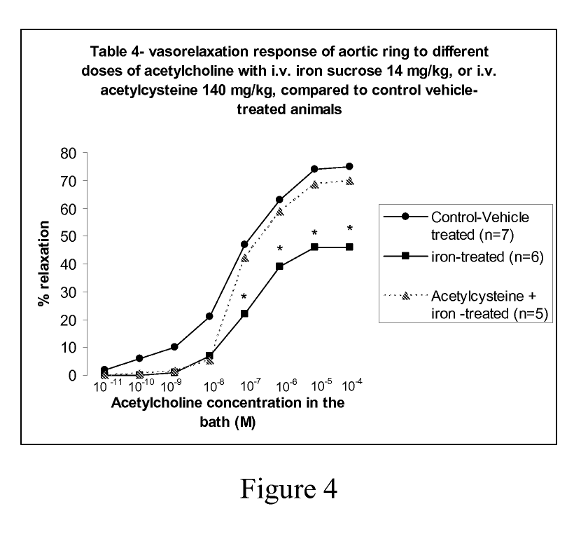

### DETAILED DESCRIPTION OF THE INVENTION

Disclosed herein are methods and compositions for the treatment of ulcers by administering a therapeutically effective amount of 4-hydroxy-2,2,6,6-tetramethyl-1-piperidine-1-oxyl (Tempol), N-acetylcysteine (NAC), or combinations thereof. It is believed that the disclosed methods and compositions provide a novel approach to treating ulcers by addressing increased blood flow to ulcers and preventing the damaging effects of hydroxyl radicals formed by free iron.

The nitroxide 4-hydroxy-2,2,6,6-tetramethyl-1-piperide-1-oxyl (a.k.a. Tempol) is an antioxidant that has demonstrated utility in the treatment of hypertension. See e.g., U.S. Pat. Nos. 6,096,759 and 6,617,337.

It is believed that the vasodilator action of Tempol will help to preserve blood flow to an ischemic region. Unlike other vasodilators, Tempol restores a normal physiologic function to blood vessels allowing myogenic and autoregulatory control of blood flow to persist. Without wishing to be bound by theory, it is believed that this is important in the skin since unopposed vasodilation might theoretically worsen the condition where pre-capillary vasoconstriction is required to prevent excessive capillary damage from the high hydrostatic pressure.

Furthermore, the present inventors have discovered that not only is Tempol a superoxide dismutase mimetic, but it also has greater sensitivity in preventing Fenton-type signaling with the formation of damaging ROS. As such, it is believed that Tempol should prevent Fenton reactions, hydroxyl radical formation, nitric oxide inactivation, and tissue damage due to hydroxyl radical formation.

Tempol is only one of a number of nitroxides that are suitable for use in treating the conditions disclosed herein. For a list of other suitable nitroxides see, for example, U.S. Patent Application Publication No. 20070021323.

N-acetylcysteine (NAC) is a relatively inexpensive and well tolerated drug. It is a delivery form of L-cysteine, which serves as a major precursor to the antioxidant glutathione. It also reduces the formation of pro-inflammatory cytokines, such as IL-9 and TNF-α. N-acetylcysteine also acts as a vasodilator by increasing cyclic GMP levels and contributing to the regeneration of nitric oxide (a.k.a. endothelial-derived relaxing factor (EDRF)).

To date, there are few studies supporting the anti-oxidant effect of NAC. N-acetylcysteine has been demonstrated to significantly decrease MDA levels in patients with stage 3 and 4 CKD within 3 to 24 hours after treatment with intravenous iron sucrose (al R. a., 2004). However, this effect was not observed in CKD patients treated with iron gluconate (al K. B., 2006).

In 2007, the present inventors demonstrated that intravenous iron sucrose injection in rats resulted in a 40% reduction in aortic ring vasodilation in response to acetylcholine administration 24 hours after the intravenous iron sucrose injection. The iron-induced endothelial dysfunction of the blood vessel was completely attenuated when iron was co-administered with NAC or Tempol (al. N. P., November 2007). As such, oral administration of Tempol and related nitroxides, N-acetylcysteine, or both is predicted to have a beneficial effect in preventing vascular and systemic oxidative stress in patients given intravenous iron as part of their therapy, preserve blood vessel function, and lessen the probability of intravascular thrombosis that can lead to heart attack, stroke, peripheral vascular disease, dementia, or the like, and prevent progression of atherosclerosis. Likewise, oral or topical administration of Tempol and related nitroxides, N-acetylcysteine, or both is predicted to have a beneficial effect on the prevention, treatment or amelioration of an ulcer.

Likewise, clinicians have established that topical forms of Tempol penetrate into the skin sufficiently to prevent the damaging effects of ionizing radiation mediated through reactive oxygen species (Metz, et al., Clinical Cancer Research 10: 6411-6417, 2004). This demonstrates that topical Tempol should reach sites of oxidative damage. In certain embodiments, methods are provided for the treatment of ulcers using 4-hydroxy-2,2,6,6-tetramethyl-1-piperidine-1-oxyl-containing composition. In particular embodiments methods are provided for the treatment of ulcers using an N-acetylcysteine-containing composition. In particular embodiments, methods are provided for the treatment of ulcers using a combination of a hydroxy-2,2,6,6-tetramethyl-1-piperidine-1-oxyl-containing composition and an N-acetylcysteine-containing composition.

In certain embodiments, the ulcer is a cutaneous ulcer. In particular embodiments, the cutaneous ulcer is an ulcer associated with one or more of the following conditions: peripheral arterial and embolic disease, leukocytoclastic vasculitis, hemoglobinopathies such as sickle cell disease, cryoglobulinemia, cholesterol emboli, necrobiosis lipoidica, antiphospholipid syndrome, neuropathies,panniculitis, Raynaud's phenomenon, pyoderma gangrenosum, calciphylaxis, infection from dimorphic fungi, chronic herpes varicella-Zoster lymphoma, Behcet's syndrome, erythema multiforme, primary blistering disorders, lupus erythematosus and inflammatory bowel disease, and venous stasis.

In other embodiments, the ulcer is a mucocutaneous ulcer. In particular embodiments, the mucocutaneous ulcer is an ulcer associated with trauma or venous stasis.

In certain embodiments, the compositions are formulated as pharmaceutical preparations. In particular embodiments, pharmaceutical preparations are formulated as pharmaceutically-acceptable salts. Lists of suitable salts are found in, for example, Remington: The Science and Practice of Pharmacy, 21st Edition (Lippincott Williams & Wilkins, 2006).

In other embodiments, pharmaceutical preparations are formulated as pharmaceutical compositions comprising one or more of the active agents or pharmaceutically-acceptable salts thereof together with a pharmaceutically-acceptable carrier. Suitable pharmaceutical carriers are described, for example, in Remington: The Science and Practice of Pharmacy, 21st Edition (Lippincott Williams & Wilkins, 2006).

In particular embodiments, the pharmaceutically-acceptable carriers further comprise excipients and auxiliaries to facilitate processing of the active compounds into formulations for delivery to the site of action. Suitable formulations for parenteral administration include aqueous solutions of the active compounds in water-soluble form; for example, water-soluble salts. Oily injection suspensions of the active compounds may also be administered. Suitable lipophilic solvents or vehicles include fatty oils, such as sesame oil or synthetic fatty acid esters (e.g., ethyl oleate or triglycerides). Aqueous injection suspensions can contain substances that increase the viscosity of the suspension. These include, for example, sodium carboxymethyl cellulose, sorbitol, and dextran. Optionally, the suspension can also contain stabilizers.

In particular embodiments, the pharmaceutical compositions are formulated for sustained delivery of the compounds in accordance with the present methods for a period of several days to a month or more. Such formulations are described, for example, in U.S. Pat. Nos. 5,968,895 and 6,180,608. Any pharmaceutically-acceptable, sustained-release formulation known in the art is contemplated.

As used herein, administering or administration includes dispensing, delivering or applying a compound disclosed herein in a method disclosed herein, e.g., in a pharmaceutical formulation, to a subject by any suitable route for delivery of that compound to the desired location in the subject. Particularly contemplated approaches include oral and topical administration.

Suitable formulations for oral administration include hard or soft gelatin capsules, pills, tablets, including coated tablets, elixirs, suspensions, syrups or inhalations and controlled release forms thereof.

Suitable formulations for topical administration include any common topical formulation such as a solution, suspension, gel, ointment or salve and the like can be employed. Preparations of such topical formulations are well described in the art of pharmaceutical formulations as exemplified, for example, by Remington : The Science and Practice of Pharmacy, 21st Edition (Lippincott Williams & Wilkins, 2006). For topical application, the formulations disclosed herein can be administered as a powder or spray, particularly in aerosol form.

As solutions containing Tempol discolor when left in sunlight, it is recommended that compositions containing them be protected from light.

It is contemplated that the compounds disclosed herein are administered in a therapeutically effective amount, i.e., an amount sufficient to achieve a desired result. An effective amount is also one in which any toxic or detrimental effects associated with administration of the compound are outweighed by the therapeutically beneficial effects. For example, effectiveness may be determined by the effect on ulcer healing or measuring indices of oxidative stress such as plasma levels of the stable oxidation product of arachidonic acid 8-isoprostane PGF2alpha, the stable oxidation product of linoleic acid, 13-hydroxydecanedioic acid, or the oxidized-to-reduced ratio of thioles such as cysteine. Effectiveness can also be assessed indirectly from brachial artery flow-induced vasodilation after a 5-minute period of forearm ischemia, or from the augmentation index derived non-invasively from pulse wave analysis, or from peak aortic blood flow velocity.

The compounds disclosed herein, and formulations thereof, can be administered to a wide variety of subjects. Subjects include humans, rats, mice, cats, dogs, non-human primates, horses, and cattle. Among the wide variety of subjects, humans are particularly contemplated.

Effective amounts of the compounds used in the methods of the invention may vary according to factors such as the disease state, age, and weight of the subject, and the ability of the compound to elicit a desired response in the subject. Dosage regimens can be adjusted to provide the optimum therapeutic response. Accordingly, dosages for the methods disclosed herein range from about 1 mg/kg/day to 100 mg/kg/day.

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

A study was undertaken to show the functional effect of intravenous iron sucrose on acetylcholine-induced relaxation in rat aortic rings in the presence or absence of Vitamin C, tempol, or N-acetylcysteine.

Forty seven normal Sprague-Dawley rats (280-350 g) were included in this study. Twenty three rats were injected in the tail vein with 14 mg/kg iron sucrose (Venofer, American Regent) twenty four hours before dissection of the aortic ring.

Of the 23 rats, 6 received no antioxidant, 6 were simultaneously given tail vein injections of 100 mg/kg Vitamin C, 5 were simultaneously given tail vein injections of 140 mg/kg N-acetylcysteine (Acetadote, Cumberland Pharmaceuticals), and 6 were given 2 mmol/L Tempol (Sigma) in their drinking water for 72 hours prior to dissection of the aortic ring. Since rats of this size normally drink 20-40 mL of fluid daily, this is equivalent to a Tempol dose of 40-80 μmol of Tempol, or about 100-300 μmol/kg/day of oral Tempol. Each subset of iron-treated animals were matched with a control group receiving only Vitamin C (n=6), N-acetylcysteine (n=5), Tempol (n=6), or saline (n=7).

Twenty four hours after iron sucrose injection, the rats were sacrificed by interperitoneal injection of 100 mg/kg Inactin. Blood was collected for measurement of markers of oxidative stress and the thoracic aorta isolated and placed in ice cold PSS.

The surrounding tissue of aorta was carefully dissected away and care taken to not damage the endothelium. The aorta was then cut into a 4-5 mm width transverse ring and then mounted isometrically in 10 mL organ bath containing oxygenated PSS warmed to 37° C. and gassed with air.

The aortic ring was suspended under a resting force of 2 xg and allowed to equilibrate for 30 minutes before administration of any agents. The bath was perfused with fresh PSS and was washed with PSS after each test response to the administered agents. The aortic ring was preconstricted with 10 nM phenylephrine prior to the addition of acetylcholine, and allowed to equilibrate for another 30 minutes.

Isometric contraction was measured on an oscillographic recorder. Subsequently acetylcholine was added to the bath in increasing doses of 1 nM to 100 μM and the relaxation response to each dose was recorded as the percentage of the drop in the starting resting force. The effects of Vitamin C, Tempol, and N-acetylcysteine were analyzed with analysis of variance (ANOVA) for measurements in each concentration of acetylcholine followed by a student t test. P<0.05 was considered significant.

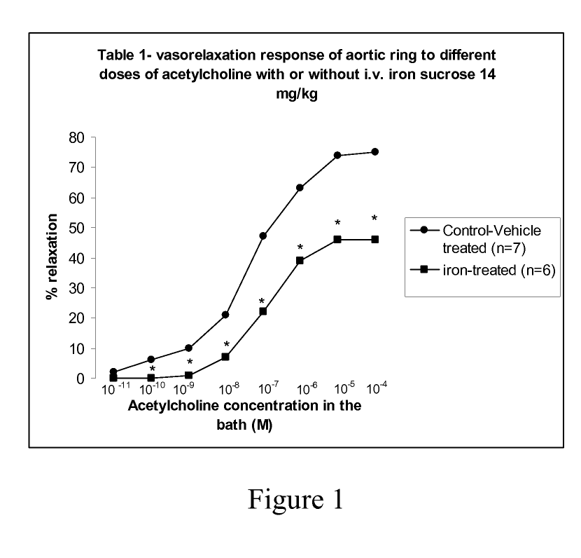

As shown in FIG. 1, the relaxation response of aortic ring to increasing doses of acetylcholine from 10 nM to 100 μM was significantly reduced with iron treatment at doses of 1×10⁻¹⁰ M and above. Maximum relaxation was achieved with 100 μM of acetylcholine and was 75±10% in control vehicle-treated rats vs. 46±14% in iron-treated animals (P<0.01).

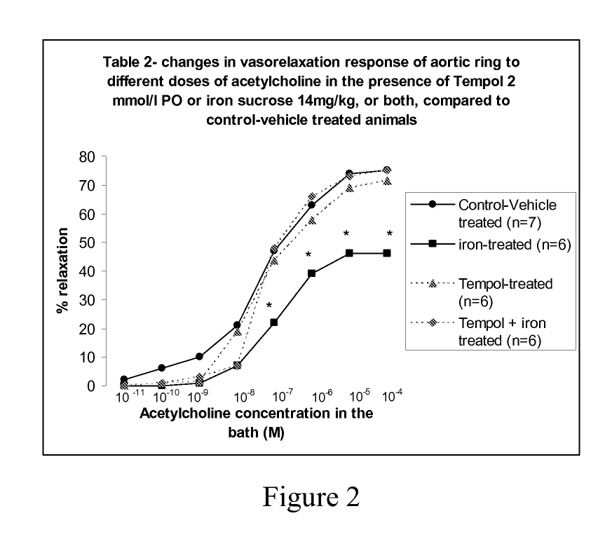

This iron-induced decrease was wholly reversed (75±15%) when iron-treated animals were fed with Tempol for 3 days prior to the study, as shown in FIG. 2 (P<0.01). The same is true when N-acetylcysteine was given in addition to iron for acetylcholine doses of 100 nM to 10 μM (max relaxation of 69.9±13%, P<0.05).

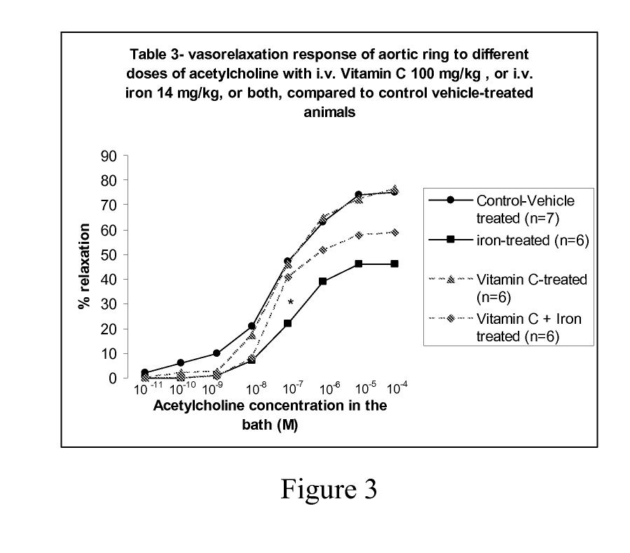

The addition of Vitamin C to iron treatment increased the relaxation response to 100 nM through 100 μM of acetylcholine but the increase was only statistically significant at the 100 nM concentration of acetylcholine (see FIG. 3).

The IC₅₀ for all dose response curves was 100 nM and the relaxation response to acetylcholine at this dose in control treated rats was 47±15%, that was significantly reduced to 22±10% in iron treated rats (P<0.01). This reduction was prevented with Tempol treatment (48±17%, P<0.01) or N-acetylcysteine treatment (42±13%, P<0.05), and Vitamin C therapy (41±8%, P<0.01) at this dose.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1.-10. (canceled)

11. A method of treating iron toxicity in a subject comprising administering a therapeutically effective amount of a nitroxide-containing composition to a subject.

12. The method of claim 11, wherein the nitroxide is 4-hydroxy-2,2,6,6-tetramethyl-1-piperidine-1-oxyl.

13. The method of claim 11, wherein the iron toxicity is associated with a condition selected from the group consisting of hemochromatosis, hemosiderosis, anemia, end-stage renal disease, or ulcer.

14. The method of claim 12, wherein the iron toxicity is associated with a condition selected from the group consisting of hemochromatosis, hemosiderosis, anemia, end-stage renal disease, or ulcer.

15. The method of claim 13, wherein the ulcer is associated with a condition selected from the group consisting of peripheral arterial and embolic disease, leukocytoclastic vasculitis, hemoglobinopathies such as sickle cell disease, cryoglobulinemia, cholesterol emboli, necrobiosis lipoidica, antiphospholipid syndrome, neuropathies, panniculitis, Raynaud's phenomenon, pyoderma gangrenosum, calciphylaxis, infection from dimorphic fungi, chronic herpes varicella-Zoster lymphoma, Behcet's syndrome, erythema multiforme, primary blistering disorders, lupus erythematosus and inflammatory bowel disease, and venous stasis.

16. The method of claim 14, wherein the ulcer is associated with a condition selected from the group consisting of peripheral arterial and embolic disease, leukocytoclastic vasculitis, hemoglobinopathies such as sickle cell disease, cryoglobulinemia, cholesterol emboli, necrobiosis lipoidica, antiphospholipid syndrome, neuropathies, panniculitis, Raynaud's phenomenon, pyoderma gangrenosum, calciphylaxis, infection from dimorphic fungi, chronic herpes varicella-Zoster lymphoma, Behcet's syndrome, erythema multiforme, primary blistering disorders, lupus erythematosus and inflammatory bowel disease, and venous stasis.

17. The method of claim 11, wherein the composition is administered orally.

18. The method of claim 11, wherein the composition is administered topically.

19. The method of claim 17, wherein the composition is administered in an amount ranging from 1-100 mg/kg/day.

20. The method of claim 18, wherein the composition is administered in an amount ranging from 1-100 mg/kg/day.

21. A method of treating iron toxicity in a subject comprising administering a therapeutically effective amount of a N-acetylcysteine-containing composition to a subject.

22. The method of claim 21, wherein the iron toxicity is associated with a condition selected from the group consisting of hemochromatosis, hemosiderosis, anemia, end-stage renal disease, or ulcer.

23. The method of claim 22, wherein the ulcer is associated with a condition selected from the group consisting of peripheral arterial and embolic disease, leukocytoclastic vasculitis, hemoglobinopathies such as sickle cell disease, cryoglobulinemia, cholesterol emboli, necrobiosis lipoidica, antiphospholipid syndrome, neuropathies, panniculitis, Raynaud's phenomenon, pyoderma gangrenosum, calciphylaxis, infection from dimorphic fungi, chronic herpes varicella-Zoster lymphoma, Behcet's syndrome, erythema multiforme, primary blistering disorders, lupus erythematosus and inflammatory bowel disease, and venous stasis.

24. The method of claim 21, wherein the composition is administered orally.

25. The method of claim 21, wherein the composition is administered topically.

26. The method of claim 24, wherein the composition is administered in an amount ranging from 1-100 mg/kg/day.

27. The method of claim 25, wherein the composition is administered in an amount ranging from 1-100 mg/kg/day.

28. A method of treating iron toxicity in a subject comprising administering a therapeutically effective amount of a 4-hydroxy-2,2,6,6-tetramethyl-1-piperidine-1-oxyl-containing composition to a subject.

29. The method of claim 28, wherein the composition is administered topically.

30. The method of claim 29, wherein the composition is administered in an amount ranging from 1-100 mg/kg/day.
